(19) United States
(12) Patent Application Publication
(10) Pub. No.: US 2002/0103505 A1
(43) Pub. Date: Aug. 1, 2002

(54) Custom manufacturing of implantable medical devices

(75) Inventor: David L. Thompson, Andover, MN (US)

Correspondence Address: Beth L. McMahon, Medtronic, Inc., MS 301, 7000 Central Avenue NE, Minneapolis, MN, 55432, US

(73) Assignee: Medtronic, Inc.

(21) Appl. No.: 09/902,016

(22) Filed: Jul. 10, 2001

Related U.S. Application Data
(63) Continuation-in-part of application No. 09/775,281, filed on Feb. 1, 2001.

(51) Int. Cl. A61N 1/00

(52) U.S. Cl. 607/1

(57) ABSTRACT

An improved system for invoicing, manufacturing, and re-programming implantable medical devices (IMDs) is disclosed. The system includes a web-enabled interface to receive manufacturing orders from remote sites such as healthcare facilities, other manufacturing sites, warehouses, and sales offices. The orders may include patient-specific information and/or requirements provided by the implanting physician or facility. For instance, patient-specific information may involve data obtained during prior patient evaluations, such as measured EKG signals and the like. This data is then used to select the software and/or hardware components to be incorporated into an IMD that is customized for the patient. For example, this data may be used to select particular software and/or hardware amplifier filters and/or digital signal processing (DSP) algorithms that may be best adapted to sense and process the unique signal characteristics associated with a patient's condition. Additionally, one or more software and/or hardware components may be selected for inclusion in the device based on optional therapies required by the patient, as determined by an implanting physician. Operating parameters may also be selected for the particular IMD. Based on the component selections, any unavailable components may be automatically ordered. Thereafter, the inventory management system may provide information to automated manufacturing and/or testing systems so that the IMD is built to order. According to another aspect of the invention, test signals generated using patient-specific data may be applied to the inputs of the manufactured IMD during test to ensure proper functioning of the customized device.

This application is a continuation in part and claims priority to U.S. patent application Ser. No. 09/775,281 filed Feb. 1, 2001, and incorporates the specification and drawings in their entireties by reference herein.

### FIELD OF THE INVENTION

The present invention relates to systems and methods of manufacturing medical devices; and more specifically, relates to an interactive manufacturing and inventory control system that derives requirements for optimizing characteristics and functions of implantable medical devices

### BACKGROUND OF THE INVENTION

Over the years, many implantable medical devices (IMDs) have been developed to monitor medical conditions and deliver therapy to a patient. Such devices included electrical stimulation devices for stimulating body organs and tissue. Stimulation may be delivered to enhance a body function or to control pain. Other implantable drug delivery devices are adapted to deliver biologically active agents at a selected site. More passive IMDs have been developed for monitoring a patient's condition.

Chronically-implanted devices for monitoring cardiovascular conditions and for providing therapies to treat cardiac arrhythmias have vastly improved the quality of life for many patients. Additionally, such IMDs have reduced mortality in patients susceptible to sudden death due to intractable, life threatening tachyarrhythmias. Examples of these types of devices include systems to process electrogram data and other measured physiological conditions. This data may be stored within the device, and may further be transferred to an external device such as a programmer using a communication system. In general, the manner of communicating between the transceivers of the external programmer and the implanted device during programming and interrogating is referred to as telemetry. U.S. Pat. Nos. 5,891,180 and 6,082,367 to Greeninger et al. describe various telemetry systems and methods for use with IMDs.

One problem associated with the use of a conventional telemetry system is that the transmitter and receiver must be within a relatively short distance of one another. For example, a programmer receiving data from an IMD must generally be located within the same room as the patient. This is viewed as unduly restrictive.

Some longer range telemetry systems are available. For example, commonly-assigned U.S. Pat. No. 5,113,869 to Nappholz, et al. describes an implanted ambulatory ECG patient monitor that provides longer range telemetry communication with a variety of external devices, including an external programmer, a remote telephonic communicator, and a personal communicator alarm. For example, the telephonic communicator may be used to establish a telephonic communication link to transmit data received from the implanted monitor to a previously designated clinic or physician's office through a modem. Similarly, the external programmer allows programming and interrogation functions to be performed from remote locations. The system thereby increases the range of communication with an implanted medical device.

Other systems are available for providing longer-range communication with implantable devices. For example, a hand-held interrogator for an implanted pacemaker-cardioverter-defibrillator device is disclosed in U.S. Pat. No. 5,336,245 to Adams et al. The interrogator transfers data from a limited-capacity memory within an implanted device to a larger capacity, external data recorder. The accumulated data is also forwarded to a clinic via an auto-dialer and FAX modem.

U.S Pat. No. 5,752,976 to Duffin, et al., incorporated herein by reference in its entirety, describes a system for transferring patient device information between an IMD implanted in an ambulatory patient and a remote medical support network. The IMD includes a transceiver that communicates with a control device located in relatively close proximity to the patient. The control device is capable of communicating with a global positioning system and with a remote medical support network. The control device is thereby able to relay patient data and location information to the remote medical support network, and also facilitate remote programming of the IMD.

As is evident from the foregoing discussion, long-range telemetry systems are valuable tools for communicating information such as patient data and/or programmable information between an external device and the IMD. Heretofore, however, such systems have not generally been used within the medical industry for inventory control and/or to customize the manufacture of IMDs to meet individual patient requirements.

Inventory control has become an important issue within the medical device industry. When dealing with the manufacture and distribution of medical devices, many unique considerations must be taken into account. For example, a customer's need for a given product must generally be satisfied very quickly, even though that need may be difficult to predict in advance. Additionally, it is important that medical inventory be consumed prior to devices becoming outdated or obsolete. Moreover, it is important that transitions to new products be managed smoothly based on FDA approvals. Finally, in some instances, it is desirable to customize a given device to the requirements of a healthcare facility, a physician, or to the needs of the patient. Given the foregoing considerations, it is difficult to maintain a balance between ensuring an adequate inventory is available to meet patient needs while also preventing costly overstocking procedures.

Current practices often do not address the particular concerns set forth above. For example, healthcare facilities are generally provided with inventory based on expected, rather than actual, usage. Because inventory levels are based on expected usage, an unforeseen need for a particular device may result in a temporary shortage. Delivering emergency shipments to cover the shortage is expensive and inconvenient. Medical procedures may have to be delayed, resulting in added health-care expenses and patient inconvenience.

Another problem occurs when a new product is being introduced. Prior to product release, adequate supplies of the new product must be available in anticipation of receiving FDA or similar government approval. Until approval is received, however, only previously-approved products may be implanted. Therefore, both old and new products must be inventoried. Moreover, following product approval, older products are generally retrieved at an economic loss to the manufacturer as the new technology gains acceptance.

Based on the foregoing problems and considerations, what is needed is an improved inventory control system. Although not currently used within the medical industry, inventory control and build-to-order systems have been readily adopted within other areas of technology. For example, build-to-order systems were developed by Dell Computer Corporation to manufacture and assemble computers tailored to the specifications of an individual customer. Using systems such as Dell4Me™, potential customers are allowed to specify system features, including type of hard drive, memory capacity, and so on. This reduces expenses by reducing the amount of inventory, personnel, and other overhead associated with the ordering and manufacture process. Systems of this nature are described in U.S. Pat. Nos. 5,894,571 and 5,995,757. Another similar system is described in U.S. Pat. No. 6,078,900 to Amberg et al., which discusses a method for estimating stock levels in production/distribution networks.

The above-discussed general-purpose inventory management systems do not address all of the needs associated with the medical device industry. For example, as mentioned above, it may be desirable to customize a given device to meet the requirements of a particular healthcare institution, a particular physician, or the specific needs of a patient.

The operation of an IMD may be tailored to meet specific requirements in a number of ways. For example, U.S. Pat. No. 4,665,919 to Mensink, incorporated herein by reference in its entirety, describes an IMD that includes one or more switchable circuits. A control system selects the operating parameters of the device using the switchable circuits. Within the context of a cardiac pacer, the system may be utilized to select parameters associated with an input amplifier, including filter settings and sensitivity during predetermined portions of the pacer cycle. Further, the operation of a circuit can be monitored over a plurality of operating cycles, with controlled switching of the circuit characteristics as a function of cumulative monitored circuit performance.

Another example of tailoring an IMD to include specific characteristics is discussed in U.S. Pat. No. 5,080,096 to Thompson et al. This patent discloses a hermetically-sealed IMD that includes a memory accessible for programming via a feedthrough. The memory may be programmed with device-specific information during the manufacturing process, such as device model and/or serial numbers, sensor data, and/or circuit trim data.

Another example of customizing IMD functions to a particular patient involves providing a customized patient alert, as may be activated upon detection of a change in health condition, or upon sufficient depletion of a battery. An exemplary voice alert system for an IMD is described in U.S. Pat. No. 5,891,180 to Greeninger et al. A similar alert system could be customized by providing a message in the patient's native language, for example.

Therefore, what is needed is an improved inventory system to manage and track the supply of medical devices. This system should support the use of physician, patient, and other data to customize devices that are tailored to individual patient, physician, and facility needs and requirements.

### SUMMARY OF THE INVENTION

This invention provides an improved system for invoicing, manufacturing, and re-programming implantable medical devices (IMDs). This inventory management system includes a web-enabled interface to receive manufacturing orders from remote systems. For example, the orders may be received from remote healthcare facilities, other manufacturing sites, warehouses, sales offices, or any other site that is web-enabled. In one embodiment, some of these orders may be generated automatically when a device is removed from the stock of a healthcare facility. For example, by scanning an encoded label of a package using a bar-code reader or other input device, an inventory system located at the healthcare facility is alerted to the depletion of inventory, and in response, places an order automatically.

In other instances, orders may be manually initiated. According to one aspect of the system, some orders may be placed manually when an employee of a healthcare facility logs onto a web page executing on the inventory management system and completes the necessary ordering information. Alternatively, an order form may be completed on a remote system and sent to the inventory management system for processing.

In any of the embodiments, the ordering information may include information that is used to customize an IMD for the patient, the implanting physician, or a particular healthcare provider. This information may include patient data obtained during a prior physical examination. For example, measured physiological waveform data such as EKG signals may be provided. Other patient-specific information may include a prescription by the implanting physician involving the inclusion of one or more optional therapies into the ordered IMD. Healthcare facilities may further make specifications associated with regional or organizational standards of care that dictate the types of therapies to be incorporated within a device.

After orders are received by the inventory management system, patient-specific data may be used to select the software and/or hardware components to be incorporated into an IMD. For example, data gathered during a patient evaluation such as EKG measurements may be used to select particular software and/or hardware amplifier filters and/or digital signal processing (DSP) algorithms that are best adapted to sense and process the unique signal characteristics of the patient. Additionally, one or more software and/or hardware components may be selected for inclusion in the device based on optional therapies required by the patient. Operating parameters may also be selected for the particular IMD. Although such parameters may require fine-tuning at the time of implant, the initial settings provide a customized starting point from which the implanting physician can work. Other hardwired or software switch settings may be selected based on the patient data. Informational data may be specified for inclusion within a storage device of the IMD, including, but not limited to, patient name and medical history, drug information, device specifics including customized operating parameters, customized shipping parameters, shipping labels, the name of the implanting institution and physician, scheduled date and/or location of implant, and a label identifying the inventory management system of the implanting institution.

After components are selected for use in the IMD as automatically determined by the inventory management system, any necessary components that are not available may be automatically ordered by the system. When all components are available, the inventory management system may transfer component and patient information to automated assembly systems and manufacturing employees so that the IMD can be built according to specification. According to another aspect of the system, patient data such as physiological waveforms measured during prior patient examinations can be utilized to generate input signals that are applied to the inputs of the manufactured device to test operations of the IMD. If desired, the software code and/or hardware operating parameters may be adjusted based on the results of the testing. After testing is complete, the manufactured device may be shipped to the desired location.

Utilizing the inventive inventory management system, the turnaround time associated with ordering and manufacturing an IMD may be reduced to several days so that inventory levels in the remote locations can be maintained at a minimum level. Moreover, the system automatically tracks status of an ordered device so that information is available to the ordering facility and/or the implanting physician via a web-enabled interface on a twenty-four hour basis. The inventory management system of the current invention minimizes inventory issues for the account, as well as for the manufacturer. In addition, it simplifies the introduction of a new product, so that product “phase out” is completed more quickly. With the attainment of these benefits, the costs to the manufacturer as well as the implanting institution can also be reduced. Additionally, devices may be “built-to-order” based on patient needs, and physician and facility requirements. Other advantages and aspects of the system will become apparent to those skilled in the art from the following description and the accompanying drawings.

### BRIEF DESCRIPTION OF THE DRAWINGS

FIG. 1 is an illustration of an implantable device in accordance with the present invention implanted within a patient, and further illustrating an external programming unit.

FIG. 2 is a perspective view of the external programming unit of FIG. 1.

FIG. 3 is a system block diagram of a system in which the invention is practiced.

FIG. 4 is a flow chart of the present invention describing the steps in the inventory-management process.

FIG. 5 is a flow diagram 100 of a process describing one embodiment of gathering user-specific data that may be utilized to customize an IMD.

FIG. 6 is a flow diagram of a second embodiment of the process described in FIG. 5.

FIG. 7 is an exemplary inventory management system.

### DETAILED DESCRIPTION OF THE DRAWINGS

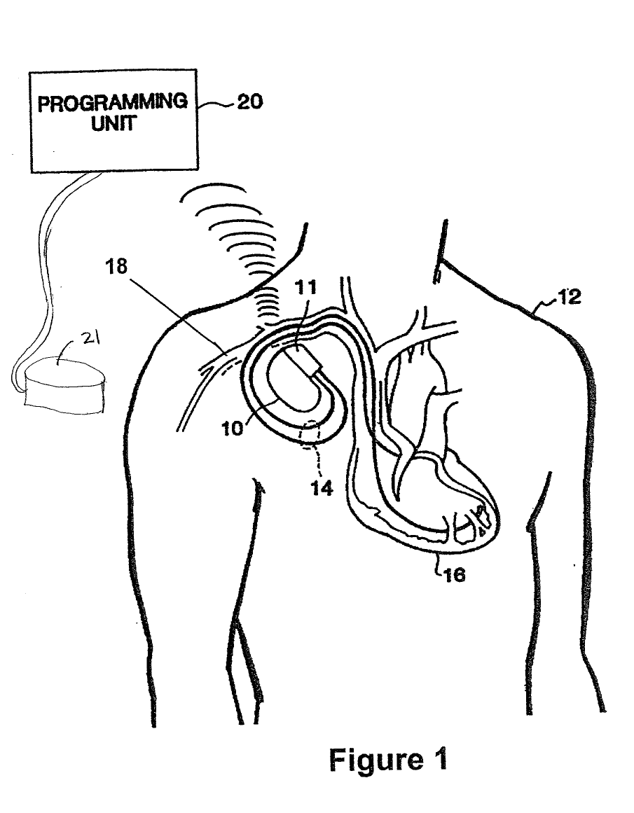

FIG. 1 is an illustration of an implantable medical device (IMD) system adapted for use in accordance with the present invention. In FIG. 1, IMD 10, which is implanted in patient 12, is shown as a pacemaker for illustration purposes. It will be understood that the present invention may be advantageously practiced in connection with numerous other types of IMDs such as cardioverter/defibrillators, drug delivery devices, neurostimulation devices, and in any application in which it is desirable to provide a communication link between two physically separated components.

In accordance with conventional practice in the art, pacemaker 10 is housed within a hermetically sealed, biologically inert outer casing, which may itself be conductive so as to serve as an indifferent electrode in the pacemaker's pacing/sensing circuit. One or more pacemaker leads, collectively identified with reference numeral 14, are electrically coupled to pacemaker 10 in a conventional manner and extend into the patient's heart 16 via a vein 18. Disposed generally near the distal end of leads 14 are one or more exposed conductive electrodes for receiving electrical cardiac signals and/or for delivering electrical pacing stimuli to heart 16. As will be appreciated by those of ordinary skill in the art, leads 14 may be implanted with their distal end(s) situated in the atrium and/or ventricle of heart 16.

Also depicted in FIG. 1 is an external programming unit 20 for non-invasive communication with implanted device 10 via uplink and downlink communication channels, to be hereinafter described in further detail. Associated with programming unit 20 is a programming head 21 in accordance with conventional medical device programming systems for facilitating two-way communication between implanted device 10 and programmer 20. Generally, programming head 21 may be positioned on the patient's body over the implant site within several inches of skin contact. One or more antennae within the head 21 can send RF signals to, and receive RF signals from, an antenna disposed within the hermetic enclosure of the implanted device or disposed within the connector block of the device, in accordance with common practice in the art. In addition, programmer 20 is also equipped with a transceiver to facilitate communication between programmer 20 and the Internet.

In one embodiment, the present invention may utilize the Global Communications and Monitoring System (GCMS) described in commonly-assigned U.S Pat. No. 5,752,976 to Duffin, et al. referenced above. In this embodiment, the implanted device includes a telemetry transceiver for communicating data and operating instructions between the implanted device and an external patient communications control device that is either worn by, or located in proximity to, the patient within the implanted device transceiving range. The control device preferably includes a communication link with a remote medical support network, and a global positioning satellite receiver for receiving positioning data identifying the global position of the control device. The control device may further include a patient activated link for permitting patient-initiated personal communication with the medical support network. The control device allows patient data and operating instructions to be exchanged between a medical support network and the IMD via a cellular telephone system link or a satellite-based telecommunications link. The GCMS is intended to function no matter how geographically-remote the patient may be relative to the monitoring site or medical support network. As such, then, during the implant procedure, with the patient is in very close proximity to the programmer, there should be no difficulty in establishing communications between the implanted device and the programmer.

Although the GCMS system may be utilized in the context of the current invention, other communication systems that support the long-range communication between an external device or system and an IMD may be used in the alternative.

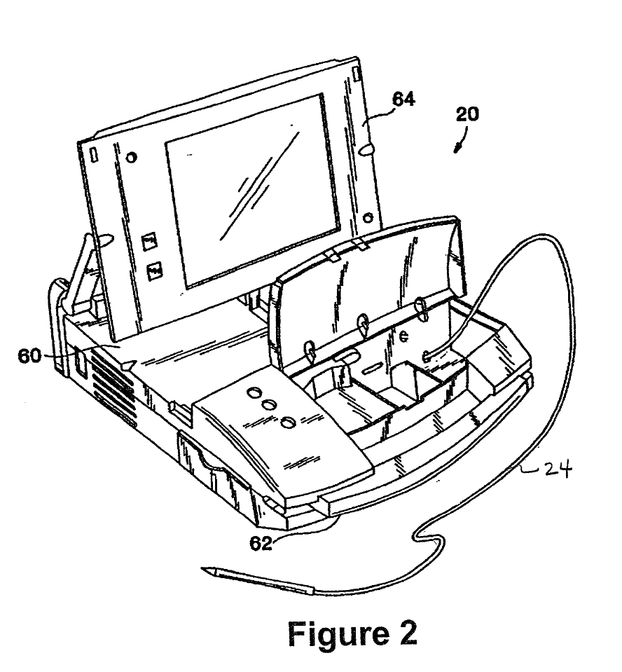

FIG. 2 is a perspective view of programming unit 20 in accordance with the presently disclosed invention. One embodiment of programmer 20 is described in commonly-assigned U.S. Pat. No. 5,345,362 to Winkler incorporated herein by reference. Similar programmers are commercially available, such as the Model 9790 programmer available from Medtronic Corporation.

Internally, programmer 20 includes a processing unit (not shown in FIG. 2), which may be a personal computer-type motherboard and related circuitry such as digital memory, although other types of general-purpose or special-purpose processing systems may be utilized. A transceiver circuit may be used to communicate data via landline or wireless communication (via telemetry) from the implanted device to a local and/or remote information network.

As is known in the art, telemetry involves communicating information via bi-directional or unidirectional electromagnetic signals such as radio frequency signals. Use of longer range telemetry systems to transfer information between IMDs and healthcare facilities is becoming increasingly important. The distance of these data transmissions may range from several yards, such as might occur within a clinical environment, or hundreds of miles, as occurs in transmission of such data between an implanting institution and an information network, as may be utilized within the context of the present invention. Wireless technology can be particularly beneficial because developing wireless networks may be faster and cheaper than building a landline infrastructure. This is discussed further below.

Returning to FIG. 2, programmer may include an outer housing 60 which is preferably made of thermal plastic or another suitably rugged yet relatively lightweight material. A carrying handle 62 may be provided to allow programmer 20 to be carried like a briefcase. Other possible features of programmer 20 may include a floppy disk drive, a hard disk drive, and/or some type of LED display to indicate system or sub-system operation status. Programmer 20 may further be equipped with an internal printer so that a hard copy of a patient's ECG can be provided. Several types of printers, such as the AR-100 printer available from General Scanning Co., are commercially available for this purpose.

Also shown in FIG. 2 is an articulating display screen 64 disposed on the upper surface of housing 60, which may be of an LCD or electroluminescent type that is characterized as being relatively thin. As would be appreciated by those of ordinary skill in the art, display screen 64 is operatively coupled to the computer circuitry disposed within housing 60 and is adapted to provide a visual display of graphics and/or data under control of the internal computer. For example, display screen may be employed to display a patient ECG or other physiological signal. Display screen 64 folds into a closed position when programmer 20 is not in use, thereby reducing the size of the system and protecting the surface of display 64 during transportation and storage.

Generally, programmer 20 will be coupled to one or more leads 24 for obtaining a patient's ECG. Such leads may be unnecessary if the IMD is equipped with a subcutaneous electrode array as described in patent application Ser. No. 09/749,169 filed Dec. 12, 2000 entitled “Leadless Fully Automatic Pacemaker Follow-Up”.

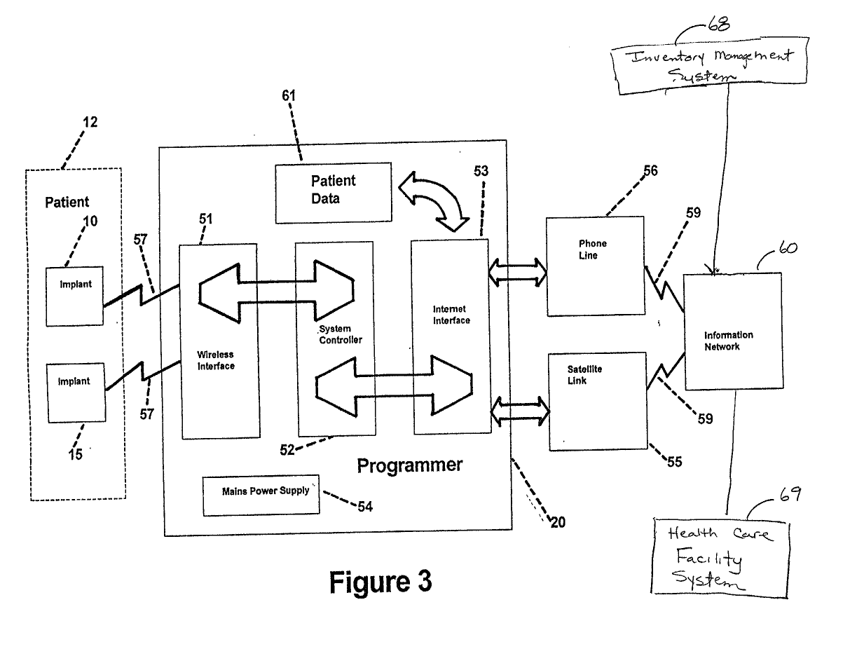

FIG. 3 is a block diagram of a system in which the current invention is practiced. The major components of the system include patient 12, programmer 20, and information network 60. Patient 12 may have multiple implants 10 and 15, which may include a bradycardia-type pacemaker and an ICD. Both devices communicate via RF link 57 to wireless interface 51 of programmer 20.

Programmer 20 is also capable of communicating with remote systems such as information network 60. This communication occurs via internet interface 53 using either phone lines 56 or satellite link 55. Data may be transferred from the information network 60 using this communication network. For example, data including factory-programmed parameters, device model numbers, serial numbers, dates of implant, and so on may be conveyed from the information network 60 to system interface 53. This data may then be stored or transmitted immediately to one or more of the implanted devices 10 and 15 via RF wireless interface 51.

Similarly, patient data from the IMDs 10 and 15 may be transferred to the information network 60 via programmer 20. This data is transferred from the IMD via RF link 57 and wireless interface 51. This information is uplinked via phone lines, cable connections, satellite links or any other type of communication network known in the art. The data transfer may utilize data encryption technology to ensure a secure transmission as substantially described in patent application Ser. No. 09/431,881, filed Nov. 2, 1999 entitled “Method and Apparatus to Secure Data Transfer from Medical Device Systems” incorporated herein by reference. Once transferred to the information network 60, this information may be incorporated into the data file containing the patient record and/or information relating to the implanting institution. This information may be utilized for diagnostic, billing, or other purposes. This data may also be utilized by an inventory management system 68, as discussed further below.

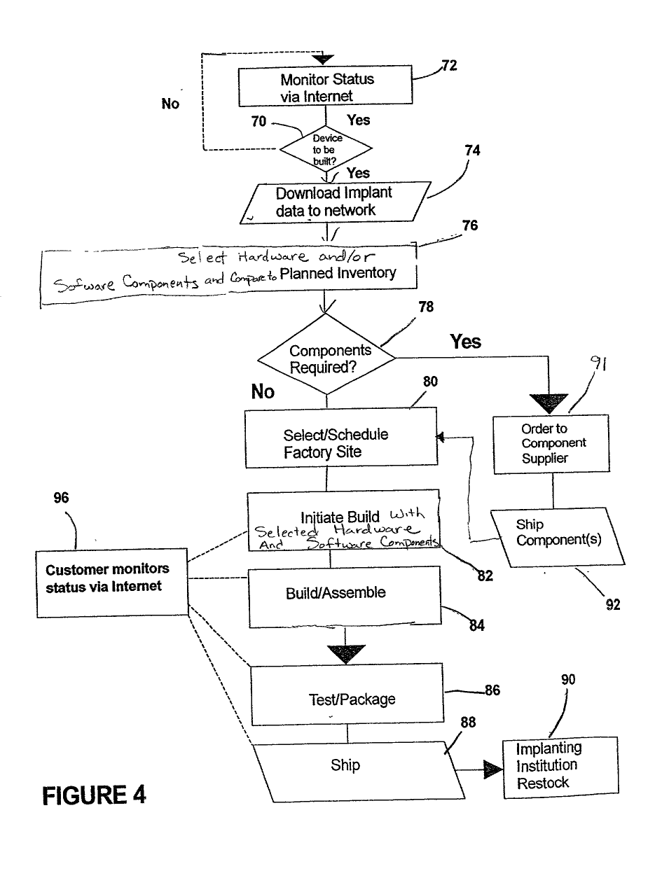

FIG. 4 is a flow chart of an inventory management system according to the current invention. The described process is designed to ensure that when an IMD has been removed from inventory because it was utilized in an implant procedure, or for any other reason such as exceeding shelf-life, the inventory supply is replaced as quickly as possible. The process is initiated in step 72, wherein inventory management system 68 is monitoring the status of the inventory. This can be accomplished, for example, by performing successive automated queries over the information network to various remote inventory systems such as those residing at hospitals and clinics, as represented by healthcare facility system 69. These queries determine if, and when, stock levels change.

Alternatively, or in addition to, the embodiment described above, status may also be provided in an unsolicited manner from one of the remote inventory systems such as that residing at healthcare facility 69 when a change in inventory occurs. This may be provided in some automated fashion, or manually. In one instance, an automated inventory control system at the remote location may send an automated request to inventory management system 68 when a particular device is removed from inventory. In another embodiment, an employee of a healthcare provider could manually enter a request to re-order a device, which would then be forwarded to the inventory management system 68 for processing. Alternatively, the employee may be allowed to sign onto the remote inventory management system to make the request using a customized web page. Other mechanisms of communicating the data to the inventory management system can be contemplated by those skilled in the art.

In any of the embodiments contemplated above, the request may include patient data associated with an up-coming implant. This data may describe optional downloadable software functions to be included within the newly-ordered device. Patient history data, drug information, implant specifics, and other data may likewise be included for downloading into a memory of the device. Physician requirements and/or preferences, as well as the requirements or restrictions of a given health care facility may be specified for consideration when manufacturing the ordered device.

After it is determined that an inventory level change has occurred, it must be determined in step 70 whether the change should result in the production of a replacement device. In some instances, it may not be desirable to replace the device. For example, a device model may be phased out over a period of time. In this instance, no replacement device is ordered, or alternatively, a different device model may be ordered. For example, a message may be sent to the ordering healthcare facility and/or physician recommending a replacement device.

If it is determined that a device is to be manufactured, processing continues with step 74, wherein the order to build, as well as any patient-specific data, is downloaded to the manufacturing database of the inventory management system 68. In step 76, the hardware and/or software needed to assemble the device is selected. The system determines which standard components are needed to manufacture the product so that it conforms with standard requirements. These standard requirements may include downloadable data such as device type, model number, serial number, the name of the implanting physician or sales representative, and the name of the implanting institution.

In a similar manner, the system determines from patient records, and physician and healthcare facility requirements, whether any custom specifications are required for this replacement. An exemplary customized data set might include, but is not limited to, specific functions and/or features of the device that may optionally be enabled or included in the software or hardware, a patient warning alarm, a voice alert in the patient's own language, customized shipping parameters, shipping labels, a patient's name and identification number, the name of the implanting institution and physician, scheduled date of implant and/or the location where that implant is to take place, and the institution's inventory management system label. Any other type of customized data could be envisioned for use within the context of the current invention. If such data is required, the system retrieves the data to be temporarily stored in member. Both the standard and custom software and data will eventually be downloaded into a storage device within the IMD during a “build-to-order” manufacturing process that produces customized device(s).

After the determination is made regarding software and hardware components to be utilized in a device, the manufacturing database will determine whether all components required to complete the build are available at the factory site located nearest to the implanting institution. This is illustrated in steps 76 and 78. If components are available, that factory site is selected and scheduled to complete the build, as shown in step 80. If components are not available, the manufacturing database issues an automatic order to the component supplier 91. The required components are noted in the database and an order to the supplier(s) is immediately initiated to secure shipment of components, as illustrated in step 92.

In steps 82 and 84, the build is initiated and completed with available components and any components delivered from the supplier. A customized device is completed to replace the implanted device in the inventory of the implanting institution. The implantable device is tested at various steps in the manufacturing process and will undergo final testing prior to packaging, as depicted in step 86. Finally, in steps 88 and 90, respectively, the device is shipped to, and re-stocked at, the implanting institution. During all of the various assembly steps, the inventory management system is tracking the assembly status. This status may be made available to the customer via the information network 60, as illustrated in step 96.

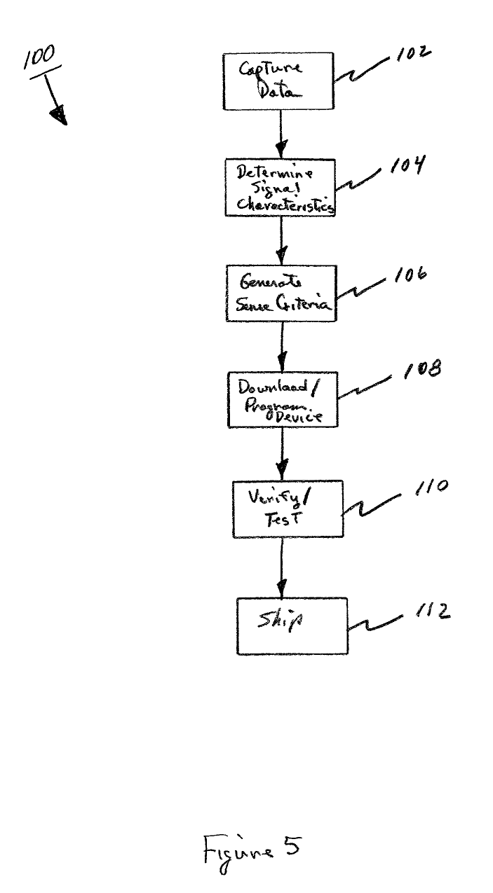

FIG. 5 is a flow diagram 100 of a process describing one embodiment of gathering user-specific data that may be utilized to customize an IMD. Patient data is gathered during a patient evaluation that may include an electro-physiology (EP) study, EKG evaluation and/or temporary pacing study, or any other type of physical analysis. This may provide information such as intrinsic and arrhythmic P- and R-waves indicative of the patient's condition. These signals are captured, identified, recorded, and stored in step 102. This data is analyzed to determine how a particular device may be customized, as shown in step 104. This analysis may be performed on the healthcare provider site, or more likely, the data will be transferred to, and analyzed on, the inventory management system 68. In particular, this data may be used to determined particular sense amplifier filter characteristics and/or digital signal processing (DSP) algorithms best adapted to sense and process the particular captured signals. This is shown in step 106. Generally, the determinations made in step 106 are utilized to select customized software for use in a particular IMD in the manner discussed above.

Mechanisms for customizing software to a patient's needs may be understood by considering known techniques for adapting algorithms after implant has occurred. For example, U.S. Pat. No. 5,447,519 to Peterson, incorporated herein by reference in its entirety, describes an implantable cardioverter/defibrillator system that is capable of discriminating between mono-morphic arrhythmias such as ventricular tachycardia and poly-morphic arrhythmias such as ventricular fibrillation. To make this distinction, an IMD includes a circuit to sample, store, and compare sets of cardiac signals to generate morphology index values that are specific for a given patient. One or more such index values may be used to distinguish between an arrhythmia and a fibrillation for a given patient. In this manner, the IMD becomes customized to a given patient after implant has occurred. Within the context of the current invention, similar techniques can be applied to previously-gathered data to tailor a given waveform analysis process to a given patient.

Another similar example is provided by U.S. Pat. No. 6,029,087 to Wohlemuth incorporated herein by reference in its entirety. This patent describes an implantable cardiac pacemaker or other cardiac monitoring system having an enhanced capability of classifying intracardiac signals through a combination of DSP techniques and software algorithms. Within the context of the current invention, the waveform morphology identification discussed in the '519 patent may be applied to the DSP techniques of the '087 patent to make the DSP processes unique to a given patient. This allows the process to more accurately differentiate between, and provide correct therapies for, intrinsic cardiac events such as ventricular mono-morphic and poly-morphic tachycardia/fibrillation, atrial tachycardia/fibrillation/flutter, sinus tachycardia, premature atrial contraction (PAC), premature ventricular contraction (PVC), left bundle branch block (LBBB), right bundle branch block (RBBB).

Yet a similar example of customizing an IMD for a particular user can be understood by considering U.S. Pat. No. 4,665,919 to Mensink referenced above. That reference describes switchable circuits to select the operating parameters of the device. Within the context of a cardiac pacer, the system may be utilized to select parameters associated with an input amplifier, including filter settings and sensitivity during predetermined portions of the pacer cycle. Further, the operation of a circuit can be monitored over a plurality of operating cycles, with controlled switching of the circuit characteristics as a function of cumulative monitored circuit performance. Within the context of the current invention, the switchable circuits that control amplification parameters may be adapted based on a patient's individual waveform characteristics. In this manner, the sense amplifier and other circuit characteristics may be optimized by selecting hardware and/or software-enabled switch setting at the time of IMD assembly.

As noted above, individual patient data obtained from prior patient examinations and studies may be utilized to select and customize software components such a DSP algorithms and software filters that are tuned for a patient's individual waveform morphology. This data may further be utilized to select customized circuit components such as amplifier components and sensing components instead of, or in addition to, the customized software.

Returning now to FIG. 5, after the appropriate software components are selected, a download of these components may be competed, as shown in step 108. The device is tested for proper function in step 110. After proper operation is verified, the device is shipped to the implanting healthcare facility in step 112 and implanted as described herein above.

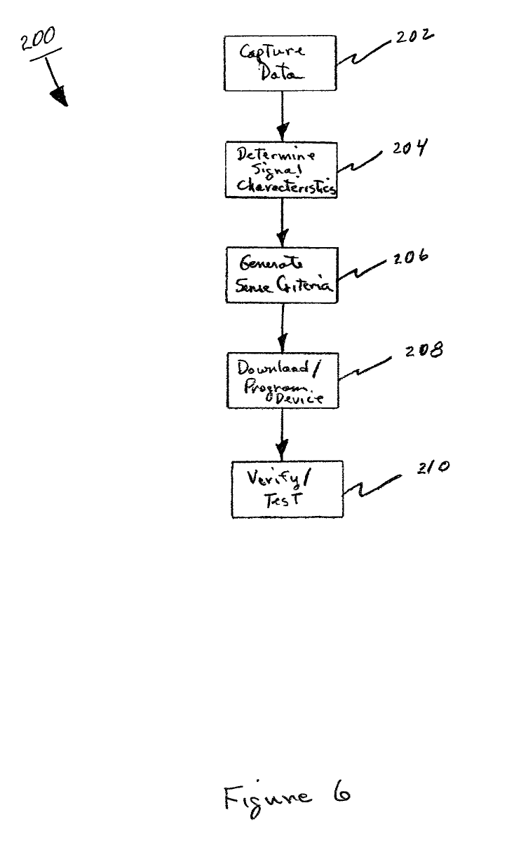

FIG. 6 is a flow diagram 200 of a second embodiment of the process described in FIG. 5. The patient data may be captured during an EP study, EKG evaluation and/or temporary pacing whereby exemplary intrinsic and arrhythmic P- and R-waves are captured, identified, recorded and stored at step 202 by programmer 20 illustrated in FIG. 3. Programmer 20 identifies and analyzes signal characteristics, as shown in step 204. Sense amplifier filter characteristics or, alternatively, digital signal processing (DSP) algorithms, are selected at step 206. The software and/or programmable parameters identified in step 206 are downloaded into the implantable medical device 10 by the programmer, as illustrated in step 208. The device may be tested for proper function in step 210 by applying the captured signal data to the input of the sense amplifier to verify proper device function.

As previously mentioned, the current invention provides an IMD which has functionality and characteristics that are optimized for a specific patient, and which may also take into consideration physician and healthcare facility requirements. Because of the web-enabled nature of the system, this can be accomplished in a very short period of time, such as within three working days of receipt of the information from the patient. Alternatively, such customized data may be configured/programmed by the implanting physician at time of implant, or during a follow-up procedure.

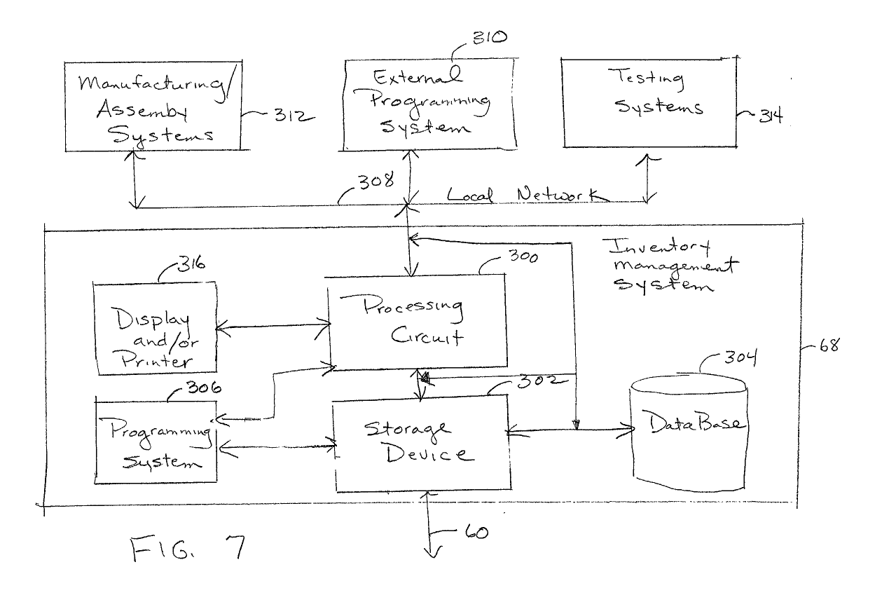

FIG. 7 is an exemplary inventory management system 68, although many other configurations are possible within the scope of the current invention. The system includes a processing circuit 300 coupled to a storage device 302. The storage device 302 includes programmable instructions executed by the processing circuit. For example, the programmable instructions may include a process for selecting software and hardware components to customize an IMD in the manner discussed above. The storage device may further store, on a temporary or a longer-term basis, one or more of the software components and/or parameters that are selected to customize an IMD. One or more of these components and/or parameters may be loaded into the storage device from another system coupled to the information network 60 such as healthcare facility system 69. In one embodiment, others of the components and/or parameters may be selected from another storage device such as database 304. Database 304 may further include information about component availability. Ordering of components could be automatically triggered via the web-enabled interface upon reaching a predetermined inventory level for a particular component that is selected for use in an IMD. Alternatively, a flag could be provided to manufacturing personnel to trigger a manual ordering procedure.

As discussed above, the inventory management system includes a web-enabled interface to the information network 60. The system may further include an interface and programming system 306 to pluggably receive one or more types of IMDs, and to download the standard and/or customized software and parameters during the assembly process. Alternatively, the system may be coupled by network such as local area network (LAN) 308 to a second external programming system 310 which receives the software and parameters to perform the programming of the devices. The inventory management system may further be coupled to other automated manufacturing/assembly systems 312 such as machines to automatically populate circuit boards with components. The inventory management system may thereby communicate any additional information needed by these systems to complete the assembly process using the correct components. Similar information could be automatically communicated to test systems 314 so that these systems may adjust test regimens based on the software and hardware components used within a particular IMD. In one embodiment, physiological signals captured from the patient during prior patient examinations may be used to generate test signals applied to inputs of the IMD such as the amplifier inputs. This verifies circuit operations and functionality.

Within the system, the manufacturing status of an IMD may be monitoring by inventory management system and provided to the healthcare facility system 69 in the manner discussed above. Information management system may further include a display device and/or printer 316 to provide a production manager or other employee with an analysis of selected components so that manual assembly steps may be performed, if necessary.

Many variations of the above system will be apparent to those skilled in the art. For example, although the current invention is described for exemplary purposes in term of implantable medical devices, it will be understood that any medical device may be manufactured and customized using the systems and processes described herein including, but not limited to, pacemakers, cardioverter-defibrillators, neurological stimulators, leads, drug delivery systems, lead adapters, and lead repair Moreover, the invention may be used for such purposes as controlling manufacturing planning and scheduling, forecasting product consumption, purchasing device components, controlling inventory at manufacturing facilities, performing vendor management, tracking materials, planning for capacity, and shipping and distributing of finished product. Furthermore, the inventory management system may receive data from other sources in addition to healthcare facilities, including, but not limited to, warehouses and sales offices.

Other expedients known to those of skill in the art or disclosed herein may be employed without departing from the invention or the scope of the appended claims. It is therefore to be understood, that within the scope of the appended claims, the invention may be practiced otherwise than as specifically described without actually departing from the spirit and scope of the present invention.

What is claimed is:

1. A system to control the configuration of an implantable medical device (IMD), comprising:
a web-enabled information network;
a storage device capable of receiving information from the information network to receive patient-specific data; and
a processing circuit coupled to the storage device to select components to be integrated in the IMD based on the patient-specific data.

2. The system of claim 1, an further including software components loaded into the storage device, the software components being selected by the processing circuit as one or more of the components selected for use in the configuration of the IMD.

3. The system of claim 2, wherein the software means is selected from the group consisting of software and/or firmware-implemented digital signal processing processes, filters, and signal differentiation processes.

4. The system of claim 3, wherein the signal differentiation processes include means to analyze cardiac waveforms selected from the group consisting of poly-morphic ventricular tachycardia, poly-morphic ventricular fibrillation, mono-morphic ventricular tachycardia, mono-morphic ventricular fibrillation, atrial flutter, atrial tachyarrhythmia, atrial fibrillation, premature atrial contractions, premature ventricular contractions, sinus tachycardia, left bundle-branch block, right bundle branch block, antegrade p-waves, and retrograde p-waves.

5. The system of claim 1, wherein the processing circuit includes means to select predetermined parameters to be downloaded into the IMD.

6. The system of claim 5, wherein the predetermined parameters are selected from the group consisting of a patient identifier, a device type, model number, a serial number, a name of an implanting physician, a name of a sales representative, a name of an implanting institution, a data of implant, a customized patient alarm, and a customized message in a selected language.

7. The system of claim 1, and further including a manufacturing system coupled to receive information indicative of the selected components, wherein the received information is used during manufacture of the IMD.

8. The system of claim 7, and further including a testing system coupled to receive information indicative of the selected components, wherein the received information is used in testing a manufactured IMD.

9. The system of claim 8, wherein the received information includes signals generated from the patient-specific data applied to inputs of the IMD during the testing of the manufactured IMD.

10. The system of claim 9, wherein the processing circuit includes means for monitoring status of an IMD being manufactured and tested, and further including means for transferring the status via the web-enabled information network to a remote system.

11. The system of claim 1, wherein the processing circuit includes means for selecting hardware components to be used during manufacture of the IMD based on the patient-specific data.

12. The system of claim 1, and further including means for monitoring inventory levels of the selected components and for ordering additional ones of the selected components when the inventory levels are within a predetermined range.

13. The system of claim 1, and further including means for receiving an order to manufacture the IMD via the web-enabled information network.

14. The system of claim 13, and further including means for automatically shipping a manufactured IMD in response to the order.

15. The system of claim 1, wherein the processing circuit is integrated within a programmer, and further comprising a telemetry system capable of downloading the ones of the selected components to the IMD.

16. The system of claim 1, and further including a programmer coupled to the storage device to download ones of the selected components to the IMD.

17. A method of utilizing an information network coupled to an inventory management system to manufacture an implantable medical device (IMD), comprising the steps of:
a.) transferring a customized order for the IMD from a remote site to the inventory management system via the information network; and
b.) utilizing the inventory management system to select a user-specific configuration of the IMD based on the customized order, the user-specific configuration to be used to manufacture the IMD.

18. The method of claim 17, wherein selecting the configuration includes selecting the operating parameters of the IMD.

19. The method of claim 17, wherein selecting the configuration includes selecting hardware components to be used in the manufacture of the IMD.

20. The method of claim 19, and further including the steps of:
determining whether the selected hardware components are available in inventory; and
automatically ordering components that are not available in inventory.

21. The method of claim 17, wherein selecting the configuration includes selecting one or more software algorithms to be used to control operations of the IMD.

22. The method of claim 21, wherein the customized order includes physiological data, and further including the step of modifying a selected software algorithm based on the physiological data.

23. The method of claim 21, wherein selecting one or more software algorithms includes selecting from software and/or firmware-implemented digital signal processing algorithms, filters, and signal differentiation algorithms.

24. The method of claim 17, wherein the customized order includes predetermined parameters selected from the group consisting of a patient identifier, a device type, model number, a serial number, a name of an implanting physician, a name of a sales representative, a name of an implanting institution, a data of implant, a customized patient alarm, and a customized message in a selected language.

25. The method of claim 17, and further including the step of using the selected configuration to manufacture the IMD.

26. The method of claim 25, wherein the inventory management system is coupled to a manufacturing system, and further including the step of transferring the selected configuration to the manufacturing system for use in manufacturing the IMD.

27. The method of claim 25, and further including the step of using the selected configuration to test a manufactured IMD.

28. The method of claim 27, wherein the inventory management system is coupled to a test system, and further including the step of transferring the selected configuration to the test system for use in testing a manufactured IMD.

29. The method of claim 27, wherein the customized order includes physiological data obtained from a patient, and further including the step of providing the physiological data to interfaces of the manufactured IMD to test the manufactured IMD.

30. The method of claim 25, and further including the step of monitoring status of an IMD while the IMD is being manufactured.

31. The method of claim 30, and further including transferring the status to the remote site.

32. The method of claim 25, and further including automatically shipping a manufactured IMD in response to the customized order.
